(19) United States
(12) Patent Application Publication
(10) Pub. No.: US 2024/0024399 A1
(43) Pub. Date: Jan. 25, 2024

(54) BIOACTIVE COMPOUNDS EXTRACTION FROM PLANT MATTERS OF SILYBUM MARIANUM AND USAGE

(71) Applicant: Munisekhar MEDASANI, Hyderabad (IN)

(72) Inventor: Munisekhar MEDASANI, Hyderabad (IN)

(21) Appl. No.: 18/028,069

(22) PCT Filed: Sep. 27, 2021
(86) PCT No.: PCT/IB2021/058777

§ 371 Date: Mar. 23, 2023

Related U.S. Application Data
(60) Provisional application No. 63/084,714, filed on Sep. 29, 2020.

(51) Int. Cl. A61K 36/28; B01D 11/02; A61K 31/7034; A61K 31/704

(52) U.S. Cl. CPC A61K 36/28; B01D 11/0288; A61K 31/7034; A61K 31/704; A61K 2236/35

(57) ABSTRACT

Lipophilic extract of plant matters of Silybum marianum containing bioactive compounds including fatty acids for use in the treatment or management of infections, disorders and diseases of liver and for use as hepatoprotective, immuno-regulator and anti-inflammatory agent.

Liver as organ is most important in detoxification of the body, elimination of toxins and waste products, taking part in production of proteins which are required for immunity. Because of obesity and diabetes about one third of the people with modern life style are getting affected with one form or other liver diseases, which are affecting over all health and becoming economic burden to individuals, family and also society.

### Prevalence of NAFLD in the Western World

In the United States, 64 million people are estimated to have NAFLD; of which, 6.65 million have NASH. There were estimated 232,000 cases of incident NASH reported in 2017 in the United States.³ In a smaller study conducted in Manitoba, Canada, incidence of NASH was reported to be between 28 and 36%. Prevalence of NAFLD in South America is 30.4%, with up to a third of these individuals progressing to NASH. Depending on the country, the prevalence of NAFLD varies from as low as 13% in Peru to as high as 30% in Brazil. Most of these studies used abdominal ultrasound and transaminases to estimate the prevalence of NAFLD. Prevalence of NAFLD diagnosed by ultrasound and liver enzymes in Italy was noted to be 23%, the Netherlands 34%, Hungary 23%, and Finland about 41%.⁷ Prevalence of NAFLD seem to be more in UK (46.2%) among all European countries compared to Germany (about 30%), Romania 20%, and Spain 25.8%. As per recent estimates, prevalence of NAFLD in the United States based on liver ultrasound in general population is around 24%. (from Ref:4)

### Epidemiology of NAFLD in Asia

Younossi et al. reviewed data from 86 studies and reported a global prevalence of NAFLD as 25.2%. The prevalence was highest in the Middle East (31.8%) and South America (30.5%) and lowest in Africa (13.5%). The prevalence of NAFLD in Asia was 27.4%, which was not lower than that in Europe (23.7%) and North American (24.1%).(from Ref:5)

Asia is a vast continent including countries with different lifestyle habits and economic development. In the same meta-analysis by Younossi et al., the prevalence of NAFLD in Asia ranged from 14.8% in Taiwan to 43.9% in China. It is important to remember that the included studies differ in research setting, study population, the method of diagnosis and the year of investigation. Studies at hospital clinics tend to report a higher prevalence of NAFLD than true general population studies. The prevalence was also higher in recent than old studies. The latter suggests that NAFLD is increasingly common in Asia. In one of the few secular trend studies, Kojima et al. reported that the prevalence increased from 12.6% in 1989 to in 1998 in Japan. Similarly, a meta-analysis of studies from China also showed an increase in NAFLD prevalence from 18.2% in 2000-2006 to 20.7% in 2010-2013.(From Ref: 5).

Above two articles indicates severity of liver diseases which need better working product besides requirement of immediate life style changes before it reaches to NASH. Fatty liver, NAFLD can be reversed with life style changes besides medication. Once it touches NASH, inflammation becomes major issues which leads to liver fibrosis, liver cirrohosis and liver cancers which cannot be reversed. This invention trying to address these issues with a innovation in the product from the existing knowledge or processes to bring out better working product with better safety profile.

Silybum marianum (Milk thistle, Silybum marianum L. Gaernt) is being used from ancient times for treatment of liver dysfunctions, gallbladder disorders. References can be found in the Old Testament (Genesis 3:18). In ancient Greece, Indian and Chinese medicines used Silybum marianum for liver and gallbladder problems. Due to its hepatoprotective use, silymarin, an extract of Silybum marianum fruits, classified by WHO as an official medicine with hepatoprotective properties.(from Ref:1)

Silymarin represents 1.5-3% of the fruit's dry weight and is an isomeric mixture of unique flavonoid complexes—flavonolignans. The main representatives of this group presented in silymarin are silybin, isosilybin, silychristin, isosilychristin, silydianin, and silimonin. The chemical composition of milk thistle fruit besides flavonolignans also include other flavonoids (such as taxifolin, quercetin, dihydrokaempferol, kaempferol, apigenin, naringin, eriodyctiol, and chrysoeriol), 5,7-dihydroxy chromone, dehydroconiferyl alcohol, fixed oil (60% linoleic acid; 30%, oleic acid; 9% palmitic acid), tocopherol, sterols (cholesterol, campesterol, stigmasterol, and sitosterol), sugars (arabinose, rhamnose, xylose, and glucose), and proteins. However, the highest concentration, comprising approximately 50-70% of the extract, is silybin, which is the major bioactive component of extract, which has been confirmed in various studies. The silybin concentrations typically found in common pharmaceutical products containing a silymarin range of 20-40%. Besides the hepatoprotective action, silybin has strong antioxidant properties and modulates a variety of cell-signaling pathways, resulting in the reduction of pro-inflammatory mediators. Silybin is also studied as a potential anticancer and chemo-preventive agent. Research performed last year demonstrates that silybin is able to inhibit serine proteases involved in the blood coagulation process, as well as reduce blood platelets' response to physiological agonists.(from Ref:1)

Silybum marianum is being sold as powder, for making tea, tincture, standardised Silymarin extract for human consumption for liver protection, diseases and disorders. Normal content of Silymarin is 1.5-3%, which they process and standardise with up to 95%.

Silymarin was first isolated in 1968 by German scientists at the University of Munich and then described and patented by the German herbal medicine manufacturer Madaus as a specific treatment “against liver diseases”. The first commercial preparation of silymarin was developed by Rottapharm/Madaus (Cologne, Germany) and complies with the analytical specifications reported in the European Pharmacopoeia January/2005 under “Milk Thistle fruit.” It is registered as a drug for liver diseases in many countries in Europe, Asia, America, Africa and Australia. Different forms, including capsules and tablets, are available with different dosages; the recommended daily dosage (depending on the commercial formulation used) is between 420 and 600 mg, and the majority of clinical trials have been conducted with a dosage of 140 mg three times a day.(From Ref:2)

Crude silymarin extract is lipophilic and poorly soluble in water, so only about 20-50% is absorbed from the gastrointestinal tract after ingestion. For this reason, formulation scientists have endeavored to improve the oral bioavailability and solubility of silymarin preparations, but the commercially available silymarin-containing products differ significantly in their content, dissolution and oral bioavailability of the active ingredient silibinin. In 1995, Rottapharm/Madaus invented a co-precipitation processing method that produced a high-quality silymarin (90-96% purity; approximately 60% of the content being silibinin) with an enhanced dissolution profile (>90% of silibinin liberated by the co-precipitate); this advanced processing method was subsequently patented in 2014 under the trade name Eurosil 85®. Most of the published clinical research on silymarin has used this standardized pharmaceutical preparation.(From Ref:2)

Dorata (From ref 3) mentioned Pressurised liquid extraction (PLE) parameters under study were solvent type (methanol, acetone and ethyl acetate), temperature (50, 75, 100, 125 and 1508 C), time (5, 10, 15 and 20 min) and the number of extraction cycles (1-5). For the PLE defatting process, n-hexane was applied as solvent, and parameters under study were temperature (50 and 1008 C) and time (5 and 10 min) of lipids removal. Acetone and ethyl acetate extracts were evaporated to dryness under vacuum and redissolved in methanol before chromatographic analysis. Still it uses multiple solvents including polar solvents and high temperatures for PLE.

In the present processes of Silymarin extraction it undergoes defatting, then extract silymarin and standardise. In that process they do multiple extractions at high temperatures with non-polar and polar solvents. Though they standardise the silymarin, this high exposure of heat and solvents could be reducing the efficacy of the product. Crude dry powders, crude extract & tinctures by polar solvents is considered less palatable due to its oily & bitter taste. Also those contain glucosides and glycosides which are polar soluble.

Present invention is to address following drawbacks of Silybum marianum extract:
1. Reducing Solvents usage by eliminating polar solvents usage in extraction
2. Reducing energy consumption by avoiding high heat processes
3. Making extract more palatable
4. Increasing the safety and efficacy of the extract for protection/treatment of Liver from Insulin resistance, inflammation, liver dysfunction, NAFLD, AFLD, NASH, Liver infections (including HCV) and liver tumours/cancers (including HCC); and for Inflammasome (including NLRP3) regulation, as chemo protectant in cancer treatment.

With this invention, inventor would like to bring additional benefit by:
retaining bioactive fat soluble components, mainly fatty acids which are being eliminated in the present commercial processes of silymarin
extract is expected to have ability to modulate non-coding RNAs, mainly microRNAs to protect and treat liver and its diseases and disorders
extract is effective even at lower doses for liver protection due to its better absorption in-spite of being liphophilic and safe at even higher doses

Inventor is anticipating and claiming following in the present invention:
A bioactive lipophilic extract for use as medicinal or nutraceutical or dietary supplement in the treatment or management of infections, disorders and diseases in human and animal subjects and for use as hepatoprotective, immuno-regulation and anti-inflammatory agent the extract comprising: one or more lipophilic components of glycoside and glucoside aglycons, polyphenols, flavonolignans. Flavonoids, steroidal compounds, steroidal compounds with side chains, terpenes and one or more fatty acids, fatty acid esters, essential oils; wherein the one or more lipophilic components collected during simplified extraction process of plant matters from Silybum marianum (Milk thistle, Silybum marianum L. Gaernt) with an organic non-polar solvent, and the extract being substantially free of sugar moieties and bitterness, wherein the extract comprises at least 70 pct by wt. of lipophilic components, and wherein an amount of the one or more aglycons and the one or more fatty acids, fatty acid esters together being more than about 30 pct by wt. of a total weight of the lipophilic components in the extract.
wherein the amount of water-soluble components in the extract do not exceed about 20% by wt. of the amount of said extract
wherein the extract is in the semisolid or liquid state at room temperature (˜22 deg C.).
Wherein the infections, disorders and diseases are infections are related to the liver like, but not limited to, viral hepatitis, non-viral hepatitis, alcoholic fatty liver disease (AFLD), non-alcoholic fatty liver disease (NAFLD), Nonalcoholic steatohepatitis (NASH), liver fibrosis, liver cirrhosis, inflammation of the liver, liver related cancers.
Where in the metabolic disorders includes diabetes, insulin resistance, obesity, dysregulation of mitochondria, liver function and immunity, and regulation of non-coding RNAs
the extract is for treatment and management of malignant and non-malignant tumours, cancers, inflammatory diseases, inflammation, neurological diseases, reactive oxygen species (ROS), NADPH modulation, age related diseases and aging.
wherein the extract is extracted using an organic non-polar solvent or its isomers selected from the group of, but not limited to, hexane, n-hexane, pentane, heptanes, petroleum ether, liquid carbon dioxide (liquid CO2), super critical carbon dioxide (SC-CO2), SCCO2 extraction using an organic non-polar co-solvent
The extract is formulated in the form of a powder, syrup, drink, tablet, caplet, softgel, capsule, nanogel, nano-particles, injections, parenterals, transdermal patches, absorbent gels, gel strips, liquid caps, gel caps.
Formulation is further added one or more additional components that provide complementary or supplementary therapeutic efficacy and/or are additional factors for nutrition, food, color, taste, texture, odor, flavor, bulk, stability and other characteristics.
Formulation is in a form suitable for administration through oral, intravenous, intramuscular, Intravesical, sub-cutaneous, peritoneal, rectal, nasal, trans-dermal, dermal, sublingual, buccal or other routes.
Formulation contains extract and other active or key ingredients in the range of 50 mg to 1000 mg per day dose.
Formulation is in the form of a food/dietary supplement, a medicine or an alternative medicine with or without added adjuvants.
The plant matter is selected from fruits, seeds, roots of Silybum marianum (Milk thistle, Silybum marianum L. Gaernt)
Extract is further added or co-administered with Glutathione, Co-enzyme Q10, Ascorbic Acid OR reduced forms/other forms of those for improved benefit to the subjects
The extract for use as an adjuvant therapy or adjuvant to the vaccine.
wherein the subject is an animal
wherein the subject is a human

Inventor has used similar processes for other products, but not for Silybum marianum. Due to shorter produce time and higher availability of Silybum marianum, this invention will be beneficial to the needy, who are presently about 20 pct of western elderly population who are suffering with one or other form of liver diseases or disorders, and aging.

Following are some of the examples/embodiments of improved extraction process. Any modifications, improvements to this invention done by persons who has skill and art will be in the scope and spirit of this invention and the same are anticipated.

### EMBODIMENT-1

SS are collected, cleaned, washed and dried. This entire process is done below 35 deg C.
SS are pulvarised to fine to coarse powder
Powder is loaded into extractor in the measured quantity
Extraction process is conducted with Super Critical CO2(SCCO2) at temperatures below and higher pressures needed.
HE is collected in collection vessel and sent for testing and formulation.

### EMBODIMENT-2

SS are collected, cleaned, washed and dried.
SS are pulvarised to fine to coarse powder and stored below 15 deg C.
Powder is loaded into extractor with filtration system in the measured quantity
Extraction process is conducted with Super Critical CO2(SCCO2) and with co-solvent at temperatures below 35 deg C. Co-solvent is selected from hexane, n-hexane or any other suitable apolar solvent.
Extract is collected in collection vessel and sent for further process to removal of co-solvent. Co-solvent is removed below 25 deg C. with ˜1 atm pressure.
Extract is collected and sent for testing and formulation.

### EMBODIMENT-3

SS are collected, cleaned, washed and dried to below 15% moisture content. This entire process is done below 25 deg C., more preferably 15 deg C.
SS are pulverised to fine to coarse powder below 15 deg C. and stored below 15 deg C. in vacuum sealed containers
Powder is loaded into extractor in the measured quantity which has filtration system
Extraction process is conducted using solvent at ˜1 atm pressure, below room temperature. Where in solvent is selected from hexane, n-hexane, Petroleum Ether 30-60 or any other suitable apolar solvent
Extract and solvent is collected in collection vessel and sent for further process to remove solvent. Solvent is removed in regulated air/nitrogen/oxygen/hydrogen atmosphere below room temperature, at ˜1 atm pressure. Where in core temperature during separation should not exceed 12 deg C.

Extract thus collected is in semisolid condition, sent for testing and formulation in controlled conditions.

### EMBODIMENT-4

SS are collected, cleaned, washed and dried. This entire process is done below 25 deg C.
SS are pulvarised to fine to coarse powder at below 25 deg C. and stored below 25 deg C. in vacuum sealed containers
Powder is loaded into extractor in the measured quantity which has with filtration system
Extraction process is conducted using solvent at temperatures below 35 deg C. at ˜1 atm pressure, where in solvent is selected from Petroleum Ether 30-60 deg C., more preferably Petroleum Ether 40-60 or any other suitable apolar solvent with low boiling point.
Extract and solvent is collected in collection vessel and sent for further process to remove solvent. Solvent is removed at temp below 25 deg C. with ˜1 atm pressure while making sure that any given point extract contact temp is maintained below 25 deg C. with ˜1 atm pressure.

Extract thus collected sent for testing and formulation in controlled conditions.

### EMBODIMENT-5

Process of bioactive lipophilic extract of Silybum marianum (Milk thistle, Silybum marianum L. Gaernt) involves following steps:
Collection of matured, dried seeds
Cleaning to remove foreign matters
Pulverising seeds into powder and loaded into extractor with filtration system
Non-Polar solvent is passed through, with or without soaking, the powder between 25-35 deg C. and between 1-5 times of powder w/v ratio (powder:solvent 1 w:1-5 v), filtered and solution is collected. Flushed with clean air/gas pressure to collect maximum possible solution. Solution contains solvent and lipophilic extract. Wherein solvent passing is through powder is 1-5 times, but more preferably single pass.
Solvent is separated from the extract between 5-35 deg C. by management of pressure, air/gas flow, moisture.
Extract which will be in semisolid form is collected and sent for testing, standardization, formulation and/or packing.

Wherever it is mentioned as SS in this application, it is understood that it meant seeds and/or fruits of Silybum marianum (Milk thistle, Silybum marianum L. Gaernt). Wherever it is mentioned for present invention as extract it is understood that it is lipophilic extract as claimed.

Processes described above also can be used for extraction of other plant materials like, but not limited to, Curcuma species (including turmeric), Coptis Root and Cortex Phellodendri (including Berberine), plants of Panax genus including Ginseng, genus Zingiber (Ginger) and other medicinal plants which are known for liver protection, immunity and anti-inflammatory activity and the same are within the scope and spirit of this invention.

Description/Specifications/Claims and variations described above are feasible by persons skilled in the art to make minor modifications and use as innovation without real added benefit to humans or animals and the same are within the scope and spirit of this invention.

### REFERENCES

1. Michal Bijak, Molecules. 2017 November; 22(11): 1942. (doi: 10.3390/molecules22111942)
2. Gillessen et al, Adv Ther 37, 1279-1301 (2020). doi.org/10.1007/s12325-020-01251-y)
3. Dorota W et al, Journal of Chromatographic Science 2014; 1-7 (doi:10.1093/chromsci/bmu049)
4. Sarnji N S, Verma R, Satapathy S K. Magnitude of Nonalcoholic Fatty Liver Disease: Western Perspective. J Clin Exp Hepatol. 2019; 9(4):497-505. doi:10.1016/j.jceh.2019.05.001
5. Ching-Yeung Yu B, Kwok D, Wong VW. Magnitude of Nonalcoholic Fatty Liver Disease: Eastern Perspective. J Clin Exp Hepatol. 2019; 9(4):491-496. doi:10.1016/j.jceh.2019.01.007
6. Porwal O et al, Silybum marianum (Milk Thistle): Review on Its chemistry, morphology, ethno medical uses, phytochemistry and pharmacological activities, Journal of Drug Delivery and Therapeutics. 2019; 9(5):199-206http://dx.doi.org/10.22270/jddt.v9i5.3666
7. U.S. Pat. No. 4,368,195
8. U.S. Pat. No. 4,871,763

1. A bioactive lipophilic extract for use as medicinal or nutraceutical or dietary supplement in the treatment or management of infections, disorders and diseases in human and animal subjects and for use as hepatoprotective, immuno-regulation and anti-inflammatory agent the extract comprising: one or more lipophilic components of glycoside and glucoside aglycons, polyphenols, flavonolignans, Flavonoids, steroidal compounds, steroidal compounds with side chains, terpenes and one or more fatty acids, fatty acid esters, essential oils; wherein the one or more lipophilic components collected during simplified extraction process of plant matters from Silybum marianum (Milk thistle, Silybum marianum L. Gaernt) with an organic non-polar solvent, and the extract being substantially free of sugar moieties and bitterness, wherein the extract comprises at least 70 pct by wt. of lipophilic components, and wherein an amount of the one or more aglycons and the one or more fatty acids, fatty acid esters together being more than about 30 pct by wt. of a total weight of the lipophilic components in the extract.

2. The extract as claimed in claim 1, wherein the amount of water-soluble components in the extract do not exceed about 20% by wt. of the amount of said extract

3. The extract as claimed in claim 1, where in the extract is in the semisolid or liquid state at room temperature (˜22 deg C.).

4. The extract as claimed in claim 1, wherein the infections, disorders and diseases are infections are related to the liver like, but not limited to, viral hepatitis, non-viral hepatitis, alcoholic fatty liver disease (AFLD), non-alcoholic fatty liver disease (NAFLD), Nonalcoholic steatohepatitis (NASH), liver fibrosis, liver cirrhosis, inflammation of the liver, liver related cancers.

5. The extract as claimed in claim 1, wherein the metabolic disorders includes diabetes, insulin resistance, obesity, dysregulation of mitochondria, liver function and immunity, and regulation of non-coding RNAs.

6. The extract as claimed in claim 1, wherein the extract is for treatment and management of malignant and non-malignant tumours, cancers, inflammatory diseases, inflammation, neurological diseases, reactive oxygen species (ROS), NADPH modulation, age related diseases and aging.

7. The extract as claimed in claim 1, wherein the extract is extracted using an organic non-polar solvent or its isomers selected from the group of, but not limited to, hexane, n-hexane, pentane, heptanes, petroleum ether, liquid carbon dioxide (liquid CO2), super critical carbon dioxide (SCCO2), SCCO2 extraction using an organic non-polar co-solvent

8. The extract as claimed in claim 1, is formulated in the form of a powder, syrup, drink, tablet, caplet, softgel, capsule, nanogel, nano-particles, injections, parenterals, transdermal patches, absorbent gels, gel strips, liquid caps, gel caps.

9. Formulation as claimed in claim 8 is further added one or more additional components that provide complementary or supplementary therapeutic efficacy and/or are additional factors for nutrition, food, color, taste, texture, odor, flavor, bulk, stability and other characteristics.

10. Formulation as claimed in claim 8 is in a form suitable for administration through oral, intravenous, intramuscular, Intravesical, sub-cutaneous, peritoneal, rectal, nasal, trans-dermal, dermal, sublingual, buccal or other routes.

11. Formulation as claimed in claim 8 contains extract and other active or key ingredients in the range of 50 mg to 1000 mg per day dose.

12. Formulation as claimed in claim 8, formulation is in the form of a food/dietary supplement, a medicine or an alternative medicine with or without added adjuvants.

13. The plant matter as claimed in claim 1, is selected from fruits, seeds, roots of Silybum marianum (Milk thistle, Silybum marianum L. Gaernt)

14. Extract as claimed in claim 1 is further added or co-administered with Glutathione, Co-enzyme Q10, Ascorbic Acid OR reduced forms/other forms of those for improved benefit to the subjects

15. The extract as claimed in claim 1, for use as an adjuvant therapy or adjuvant to the vaccine.

16. As claimed in claim 1, wherein the subject is an animal

17. As claimed in claim 1, wherein the subject is a human

18. Process of bioactive lipophilic extract of Silybum marianum (Milk thistle, Silybum marianum L. Gaernt) involves following steps:
Collection of matured, dried seeds
Cleaning to remove foreign matters
Pulverising seeds into powder and loaded into extractor with filtration system
Non-Polar solvent is passed through, with or without soaking, the powder between and between 1-5 times of powder w/v ratio (powder:solvent 1 w:1-5 v), filtered and solution is collected. Flushed with clean air/gas pressure to collect maximum possible solution. Solution contains solvent and lipophilic extract.
Solvent is separated from the extract between 5-35 deg C. by management of pressure, air/gas flow, moisture.
Extract which will be in semisolid form is collected and sent for testing, standardization, formulation and/or packing.
